(19) United States
(12) Patent Application Publication
(10) Pub. No.: US 2020/0215051 A1
(43) Pub. Date: Jul. 9, 2020

(54) NEBULIZATION COMPOSITION COMPRISING TIOTROPIUM AND INDACATEROL

(71) Applicant: GLENMARK SPECIALTY S.A., La Chaux-de-Fonds (CH)

(72) Inventors: Sushrut Kulkarni, Navi Mumbai (IN); Sunil Chaudhari, Nashik (IN); Suresh Rajurkar, Nashik (IN); Chetan Yewale, Dhule (IN); Rajesh Ankam, Nashik (IN); Ulhas Dhuppad, Nashik (IN)

(21) Appl. No.: 16/732,589

(22) Filed: Jan. 2, 2020

(30) Foreign Application Priority Data
Jan. 3, 2019 (IN) 201921000273
Oct. 11, 2019 (IN) 201921041250

(51) Int. Cl. A61K 31/4704; A61K 31/439; A61K 9/08; A61K 9/00; A61K 47/02; A61K 47/12

(52) U.S. Cl. CPC A61K 31/4704; A61K 31/439; A61K 47/12; A61K 9/0078; A61K 47/02; A61K 9/08

(57) ABSTRACT

This disclosure relates to a nebulization composition comprising tiotropium, or a pharmaceutically acceptable salt thereof, and indacaterol, or a pharmaceutically acceptable salt thereof. This disclosure also relates to a process for preparing such a composition and to methods of treating inflammatory and/or obstructive airway diseases using such a composition.

### CROSS REFERENCE TO RELATED APPLICATIONS

This application claims priority to Indian Provisional Patent Application No. 201921000273 filed on Jan. 3, 2019 and Indian Provisional Patent Application No 201921041250 filed on Oct. 11, 2019, the entire contents of which are incorporated by reference herein.

### FIELD OF THE INVENTION

The present invention relates to a nebulization composition comprising tiotropium, or a pharmaceutically acceptable salt thereof, and indacaterol, or a pharmaceutically acceptable salt thereof. The present invention also relates to a process for preparing such a composition and to methods of treating inflammatory and/or obstructive airway diseases using such a composition.

### BACKGROUND OF THE INVENTION

Respiratory disorders include a number of airway diseases. Asthma and chronic obstructive airway disease (COPD) are among the most prevalent and life threatening conditions.

COPD is a chronic disorder characterized by loss of elasticity of the airways and air sacs, destruction of alveolar walls, inflammation of airways, and high mucus production in the airways. All of these effects lead to clogging of the airways making it difficult for the patient to breathe. Asthma, on the other hand, is a chronic disease involving airways of the lung characterized by coughing, wheezing, and shortness of breath.

Tiotropium bromide monohydrate ((1α,2β,4β,5α,7β)-7-[(hydroxydi-2-thienylacetyl)oxy]-9,9-dimethyl-3-oxa-9-azoniatricyclo[3.3.1.0^{2,4}]nonane bromide monohydrate) is commercially marketed in the United States as capsules (Spiriva®) containing lactose monohydrate and 18 μg tiotropium (equivalent to 22.5 μg tiotropium bromide monohydrate) and as an inhalation solution (Spiriva® Respimat) containing tiotropium bromide, water for injection, edetate disodium, benzalkonium chloride and hydrochloric acid for the long-term, once-daily, maintenance treatment of bronchospasm associated with COPD and for reducing COPD exacerbations.

U.S. Publication No. 2004/0019073 discloses an aerosol formulation of a pharmaceutically acceptable salt of tiotropium dissolved in water.

A new category of active agents includes ultra-long acting beta adrenoreceptor agonists (LABAs), which typically act for about 24 hours and thereby provide the ease of once daily dosing. A newer ultra LABA in this class is indacaterol. Indacaterol, the chemical name of which is 5-[(1R)-2-[(5,6-diethyl-2,3 -dihydro-1H-inden-2-yl)amino]-1-hydroxyethyl]-8-hydroxy-1H-quinolin-2-one, is described in U.S. Pat. No. 6,878,721. The use of indacaterol for the treatment for COPD is described in U.S. Pat. No. 8,067,437. Indacaterol is currently approved in the United States as a dry powder inhaler (Aracpta Neohaler®) and as a dry powder inhaler in combination with glycopyrrolate (Utibron®). Indacaterol is indicated for the long term, maintenance treatment of airflow obstruction in patients with chronic obstructive pulmonary disease (COPD).

Indacaterol is characterized by low aqueous solubility, classified as “very slightly soluble in water.” This poses a serious challenge to solubilizing indacaterol.

International Publication No. WO 2015/018800 discloses inhalable particles comprising an intimate mixture which consists of an amorphous tiotropium compound, an amorphous indacaterol compound, and a sugar derivative.

International Publication No. WO 2019/142214 discloses a pharmaceutical composition comprising tiotropium or a pharmaceutically acceptable salt thereof and long acting beta adrenergic agonists or its pharmaceutically acceptable salts thereof for inhalation via nebulization to a subject.

There remains a need for improved treatments for respiratory disorders.

### SUMMARY OF THE INVENTION

In one aspect, the present invention provides a nebulization composition comprising tiotropium, or a pharmaceutically acceptable salt thereof, and indacaterol, or a pharmaceutically acceptable salt thereof.

In one embodiment, the present invention provides a sterile pharmaceutical nebulization composition comprising tiotropium, or a pharmaceutically acceptable salt thereof, and indacaterol, or a pharmaceutically acceptable salt thereof. In one embodiment, the tiotropium or pharmaceutically acceptable salt thereof, and indacaterol, or pharmaceutically acceptable salt thereof are present in the nebulization composition in solubilized form. In another embodiment, the nebulization composition containing tiotropium or pharmaceutically acceptable salt thereof, and indacaterol or pharmaceutically acceptable salt thereof is a suspension. The nebulization composition may be contained within a pre-filled container. The nebulization composition may be administered to relieve a subject suffering from COPD or asthma.

In another embodiment, a sterile pharmaceutical composition is a unit dose nebulizable composition comprising tiotropium, or a pharmaceutically acceptable salt thereof and indacaterol, or a pharmaceutically acceptable salt thereof. The sterile pharmaceutical composition may be administered in nebulized form to relieve bronchospasm in a subject, such as a subject suffering from COPD or asthma.

In another embodiment, a sterile pharmaceutical composition is a multi-dose nebulizable composition comprising tiotropium, or a pharmaceutically acceptable salt thereof and indacaterol, or a pharmaceutically acceptable salt thereof. The sterile pharmaceutical composition may be administered in nebulized form to relieve bronchospasm in a subject, such as a subject suffering from COPD or asthma.

Another embodiment relates to a nebulization composition comprising a combination of tiotropium, or a pharmaceutically acceptable salt thereof, and indacaterol, or a pharmaceutically acceptable salt thereof, for the treatment of an inflammatory or obstructive airway disease.

In a preferred embodiment, any of the nebulization compositions described herein comprise tiotropium bromide and indacaterol maleate.

In a further embodiment, any of the nebulization compositions described herein comprises tiotropium, or a pharmaceutically acceptable salt thereof, and indacaterol, or a pharmaceutically acceptable salt thereof in a weight ratio of tiotropium:indacaterol from about 1:0.1 to about 1:500, such as from about 1:0.2 to about 1:100.

In one embodiment, any of the nebulization compositions described herein comprises from about 5 to about 100 mcg tiotropium or pharmaceutically acceptable salt thereof.

In one embodiment, any of the nebulization compositions described herein comprises from about 10 to about 500 mcg indacaterol or pharmaceutically acceptable salt thereof.

In an embodiment, any of the nebulization compositions described herein is a sterile, unit dose composition.

In an embodiment, any of the nebulization compositions described herein is a sterile, multi-dose composition.

One embodiment of the present invention is a 0.1 ml to 6 ml nebulization composition comprising tiotropium, or a pharmaceutically acceptable salt thereof (such as tiotropium bromide) and indacaterol, or a pharmaceutically acceptable salt thereof (such as indacaterol maleate).

In one embodiment, any of the nebulization compositions described herein is in a single chamber container for administration to a patient.

In another embodiment, the tiotropium and indacaterol are contained in separate chambers of a dual chamber container. The two are combined together before, or at the time of, administration to a patient to form a nebulization composition as described herein. In one embodiment, one chamber includes a nebulization composition containing tiotropium or a pharmaceutically acceptable salt thereof (such as tiotropium bromide) as described herein but without indacaterol or a pharmaceutically acceptable salt thereof, and the other chamber includes a nebulization composition containing indacaterol or a pharmaceutically acceptable salt thereof (such as indacaterol maleate) as described herein but without tiotropium or a pharmaceutically acceptable salt thereof.

In yet another embodiment, any of the nebulization compositions described herein comprises (i) tiotropium, or a pharmaceutically acceptable salt thereof (such as tiotropium bromide), (ii) indacaterol, or a pharmaceutically acceptable salt thereof (such as indacaterol maleate), (iii) a buffering and/or pH adjusting agent, and (iv) an isotonicity agent. The nebulization compositions described herein may optionally further include a chelating agent, a pharmaceutically acceptable surfactant, a pharmaceutically acceptable vehicle, or any combination of any of the foregoing.

In one embodiment, any of the nebulization compositions described herein is free, or substantially free, of a preservative (such as a benzalkonium salt, e.g., benzalkonium chloride).

In another embodiment, any of the nebulization compositions described herein is free, or substantially free, of a complexing agent (such as ethylene diamine tetra-acetic acid (EDTA), or a salt thereof).

In a further embodiment, any of the nebulization compositions described herein is free, or substantially free, of (a) EDTA or a salt thereof and (b) a preservative such benzalkonium chloride.

Another embodiment is a prepackaged, sterile, premixed, premeasured nebulization composition comprising tiotropium, or a pharmaceutically acceptable salt thereof (such as tiotropium bromide) and indacaterol, or a pharmaceutically acceptable salt thereof (such as indacaterol maleate). Preferably, the nebulization composition is a ready-to-use dosage form which does not require any mixing or dilution by the subject prior to administration. The nebulization composition may be administered for the relief of bronchospasm in a subject suffering from COPD or asthma.

In one embodiment, the nebulization composition comprises about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and monosodium citrate, a complexing agent selected from edetate disodium, and a preservative selected from benzalkonium chloride. The nebulization composition may be in a prefilled container.

Yet another embodiment is a prefilled container containing an aqueous nebulization composition comprising (or consisting of, or consisting essentially of), per mL of composition, (i) from about 2.5 to about 20 μg of tiotropium bromide, (ii) from about 5 to about 150 μg of indacaterol maleate, (iii) 0.2 to 0.9% w/v sodium chloride, (iv) optionally up to 0.02% w/v benzalkonium chloride (e.g., at 0.01% or 0.02% w/v), and (v) optionally a buffer. In one embodiment, the buffer is a combination of tartaric acid and sodium tartrate (e.g., sodium tartrate dihydrate). In another embodiment, the buffer is a combination of phosphoric acid and sodium dihydrogen phosphate (e.g., sodium dihydrogen phosphate dihydrate). In yet another embodiment, the buffer is a combination of tartaric acid and monosodium citrate. In yet another embodiment, the buffer is a combination of citric acid (e.g., citric acid monohydrate) and trisodium citrate (e.g., trisodium citrate dihydrate). In one embodiment, the buffer is at a total content of 1%, 0.5%, 0.2%, or 0.1% w/v. In yet another embodiment, the composition is free of buffer. In one embodiment, the composition includes about 0.2 to about 0.5% w/v sodium chloride, such as 0.3%, 0.4%, or 0.5% w/v sodium chloride. The pH of the composition is preferably from about 2.5 to about 3.0. In one embodiment, the composition is free of ascorbic acid, citric acid, EDTA, and salts thereof.

After storage in the prefilled container (either an high-density polyethylene (HDPE) bottle or a glass bottle) at 25±2° C. and 60±5% relative humidity for 1 month, the content of Impurity A of tiotropium is preferably less than 0.5%, 0.4% 0.2%, or 0.1% (based on the initial amount of tiotropium bromide), the total impurities from tiotropium is preferably less than 1.5%, 1%, 0.5%, 0.4% 0.2%, or 0.1% (based on the initial amount of tiotropium bromide), the content of the monoethyl impurity of indacaterol (i.e., 5-((1R)-2-((5-ethyl-2,3-dihydro-1H-inden-2-yl)amino)-1-hydroxyethyl)-8-hydroxyquinolin-2(1H)-one) is less than 0.5%, 0.4% 0.2%, or 0.1% (based on the initial amount of indacaterol maleate), the total impurities from indacaterol is preferably less than 1.5%, 1%, 0.5%, 0.4% 0.2%, or 0.1% (based on the initial amount of indacaterol maleate), or any combination of any of the foregoing.

After storage in the prefilled container (either an HDPE bottle or a glass bottle) at 40±2° C. and 75±5% relative humidity for 1 month, the content of Impurity A of tiotropium is preferably less than 1%, 0.75%, 0.6%, 0.5%, 0.4% 0.2%, or 0.1% (based on the initial amount of tiotropium bromide), the total impurities from tiotropium is preferably less than 1.5%, 1%, 0.5%, 0.4% 0.2%, or 0.1% (based on the initial amount of tiotropium bromide), the content of the monoethyl impurity of indacaterol is less than 0.5%, 0.4% 0.2%, or 0.1% (based on the initial amount of indacaterol maleate), the total impurities from indacaterol is preferably less than 1.5%, 1%, 0.5%, 0.4% 0.2%, or 0.1% (based on the initial amount of indacaterol maleate), or any combination of any of the foregoing.

After storage in the prefilled container (either an HDPE bottle or a glass bottle) at 2-8° C. relative humidity for 1 month, the content of Impurity A of tiotropium is preferably less than 0.5%, 0.4% 0.2%, or 0.1% (based on the initial amount of tiotropium bromide), the total impurities from tiotropium is preferably less than 1.5%, 1%, 0.5%, 0.4% 0.2%, or 0.1% (based on the initial amount of tiotropium bromide), the content of the monoethyl impurity of indacaterol is less than 0.5%, 0.4% 0.2%, or 0.1% (based on the initial amount of indacaterol maleate), the total impurities from indacaterol is preferably less than 1.5%, 1%, 0.5%, 0.4% 0.2%, or 0.1% (based on the initial amount of indacaterol maleate), or any combination of any of the foregoing.

Another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises a isotonicity agent selected from sodium chloride, a buffering agent selected from tartaric acid and monosodium citrate, and a preservative selected from benzalkonium chloride.

One embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and monosodium citrate, and a complexing agent selected from edetate disodium.

A further embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and a buffering agent selected from tartaric acid and monosodium citrate.

One embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and sodium tartrate dihydrate, a complexing agent selected from edetate disodium, and a preservative selected from benzalkonium chloride.

Another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and sodium tartrate dihydrate, and a preservative selected from benzalkonium chloride.

An additional embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises a isotonicity agent selected from sodium chloride, a buffering agent selected from tartaric acid and sodium tartrate dihydrate, and a complexing agent selected from edetate disodium.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and a buffering agent selected from tartaric acid and sodium tartrate dihydrate.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, a complexing agent selected from edetate disodium, and a preservative selected from benzalkonium chloride.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, and a preservative selected from benzalkonium chloride.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, and a complexing agent selected from edetate disodium.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and a buffering agent selected from phosphoric acid and sodium dihydrogen phosphate dihydrate.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a complexing agent selected from edetate disodium, a preservative selected from benzalkonium chloride and an acid as pH adjusting agent.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, an acid as pH adjusting agent, and a preservative selected from benzalkonium chloride.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, an acid as pH adjusting agent, and a complexing agent selected from edetate disodium.

Yet another embodiment is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and an acid as pH adjusting agent.

Yet another embodiment is one or more prefilled containers containing a nebulization composition according to any of the embodiments described herein. In one embodiment, each container comprises a single unit dose of a nebulization composition according to any of the embodiments described herein for the treatment of COPD or asthma. In one embodiment, each container includes a sterile, premixed, premeasured, aqueous solution or suspension comprising a single unit dose of a therapeutically effective amount of tiotropium, or a pharmaceutically acceptable salt thereof (such as tiotropium bromide) and indacaterol, or a pharmaceutically acceptable salt thereof (such as indacaterol maleate) in a single container.

Yet another embodiment is one or more prefilled containers containing a nebulization composition according to any of the embodiments described herein wherein the composition is in a solid dosage form, e.g., a powder form, which can be reconstituted prior to nebulization with an appropriate diluent supplied in another prefilled container.

Another embodiment is a kit comprising a nebulizer and a nebulization composition according to any of the embodiments described herein. In one preferred embodiment, the nebulization composition comprises tiotropium, or a pharmaceutically acceptable salt thereof (such as tiotropium bromide) and indacaterol, or a pharmaceutically acceptable salt thereof (such as indacaterol maleate).

Another aspect of the present invention is a method of increasing the forced expiratory volume (FEV1) values in a patient by administering to the patient a nebulization composition according to any of the embodiments described herein.

Yet another embodiment is a kit and/or system for administering a bronchodilator to relieve bronchospasm, for instance, bronchospasm associated with COPD or asthma. The kit and/or system may comprise a nebulization composition according to any of the embodiments described herein. In one embodiment, the kit and/or system comprises a nebulization composition comprising tiotropium, or a pharmaceutically acceptable salt thereof (such as tiotropium bromide) and indacaterol, or a pharmaceutically acceptable salt thereof (such as indacaterol maleate) in a prepackaged, premeasured, premixed and/or single unit dose form for the treatment of COPD or asthma. In another embodiment, the prepackaged inhalation kit and/or system comprises one or more premixed, premeasured single unit dose vials comprising a nebulization composition according to any of the embodiments described herein, e.g., a composition comprising a therapeutically effective amount of tiotropium, or a pharmaceutically acceptable salt thereof (such as tiotropium bromide) and indacaterol, or a pharmaceutically acceptable salt thereof (such as indacaterol maleate) for the treatment of bronchospasm (such as that associated with COPD or asthma), and instructions for using the same.

Another embodiment is a kit comprising a nebulizer, instructions for using the nebulizer, and a unit dose vial(s) containing a nebulization composition according to any of the embodiments described herein. The time taken to administer the nebulization compositions described herein may be from about 1 minute to about 10 minutes.

Yet another embodiment is a method of treating an inflammatory or obstructive airway disease by administering a nebulization composition according to any of the embodiments described herein. In a preferred embodiment, the nebulization composition comprises tiotropium, or a pharmaceutically acceptable salt thereof (such as tiotropium bromide) and indacaterol, or a pharmaceutically acceptable salt thereof (such as indacaterol maleate). The nebulization composition may be administered at least once daily or twice daily

Yet another embodiment is a method for improving user compliance and/or quality of life in patients with asthma or COPD, as compared to conventional treatments for asthma or COPD. The method comprises initiating treatment with a nebulization composition, or a container, kit, or system according to any of the embodiments described herein. The present invention provides a convenient, fast and reliable treatment for inflammatory and/or obstructive airway diseases, such as COPD and asthma that represents an improvement over traditional treatments.

Other objects, features and advantages of the present invention will be apparent to those of ordinary skill in the art in view of the following detailed description of the invention.

### DETAILED DESCRIPTION OF THE INVENTION

The present invention relates to a nebulization composition comprising tiotropium, or a pharmaceutically acceptable salt thereof, and indacaterol, or a pharmaceutically acceptable salt thereof. The present invention also relates to a process for preparing such a composition and to methods of treating inflammatory and/or obstructive airway diseases using such a composition.

Respiratory diseases, such as asthma and COPD, are prevalent and can be life threatening. Dosage forms for the treatment of asthma and COPD include metered dose inhalation and dry powder inhalation. Drug delivery via metered dose inhalation and dry powder inhalation require synchronization with the patient's breathing pattern and the device characteristics. This often requires the patient to be trained with the device. In contrast, nebulization does not require the patient to undergo cumbersome training. With nebulization, the patient need not synchronize his or her breathing patterns with the device, and the delivery of the drug is independent of the breathing pattern of the patient. This is especially convenient for pediatric and geriatric patients. Nebulized drugs are deposited directly into the respiratory tract and thus higher drug concentrations can be achieved in the bronchial tree and pulmonary bed with fewer adverse effects.

Tiotropium is an anticholinergic agent with specificity for muscarinic receptors. It is chemically described as (1α, 2β, 4β, 5α, 7β)-7-[(hydroxydi-2-thienylacetyl)oxy]-9,9-dimethyl-3-oxa-9-azoniatricyclo [3 .3.1.02,4] nonane, and is a synthetic, non-chiral, quaternary ammonium compound. Tiotropium has a molecular formula of C₁₉H22NO₄S₂ and the following structure:

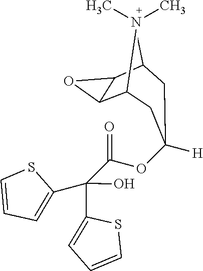 [SMILES: CC1CC(OC(=O)C(O)(c2cccs2)c2cccs2)CC(N(C)C)C2OC12]

As used herein, the term “tiotropium”, unless otherwise indicated, includes, but is not limited to, tiotropium in any physical form, such as the amorphous form and crystalline forms (e.g., anhydrous, hydrate, and other solvate forms). 2-hydroxy-2,2-dithiophen-2-ylacetic acid (referred to herein as Impurity A) is an impurity that may be present in tiotropium. Salts of tiotropium include, but are not limited to, acid addition salts and base salts thereof. Suitable pharmaceutically acceptable salts of tiotropium include, but are not limited to, halide salts such as bromide, chloride and iodide salts. These and other salts are described, for example, in U.S. Pat. No. RE 39,820, which is hereby incorporated by reference in its entirety. The preparation of tiotropium bromide monohydrate is described in U.S. Pat. No. 6,777,423, which is hereby incorporated herein by reference in its entirety. One preferred salt of tiotropium for the nebulization compositions described herein is tiotropium bromide, such as in the form of its monohydrate salt (tiotropium bromide monohydrate), anhydrous salt, amorphous salt or as an anhydrous amorphous salt.

Indacaterol maleate (5-[2-(5,6-diethylindan-2-ylamino)-1-hydroxyethyl]-8-hydroxy-1H-quinolin-2-one maleate) is a corticosteroid having a molecular weight of 508.56 and the following structure:

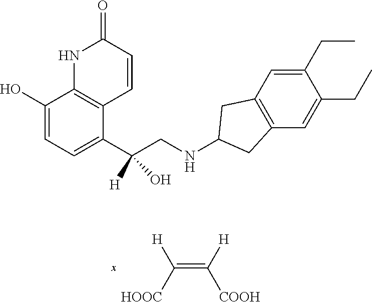 [SMILES: CCc1cc2c(cc1CC)CC(NC[C@H](O)c1ccc(O)c3[nH]c(=O)ccc13)C2 ; O=C(O)/C=C\C(=O)O]

To treat indications with a therapeutic agent, an “effective amount” of a therapeutic agent will be recognized by clinicians and persons of ordinary skill in the art, and includes an amount effective to treat, reduce, alleviate, ameliorate, eliminate or prevent one or more symptoms of the condition sought to be treated or, alternately, the condition sought to be avoided, or to otherwise produce a clinically recognizable favorable change in the condition or its effects.

### Nebulizable Compositions

In one embodiment, the nebulization compositions described herein comprise tiotropium, or a pharmaceutically acceptable salt thereof (such as tiotropium bromide) and indacaterol, or a pharmaceutically acceptable salt thereof (such as indacaterol maleate), wherein both active agents are present in solubilized form.

In another embodiment, the nebulization composition containing tiotropium, or a pharmaceutically acceptable salt thereof (such as tiotropium bromide) and indacaterol, or a pharmaceutically acceptable salt thereof (such as indacaterol maleate) is a suspension.

The nebulization compositions described herein may comprise a sterile unit dosage form comprising tiotropium, or a pharmaceutically acceptable salt thereof (such as tiotropium bromide) and indacaterol, or a pharmaceutically acceptable salt thereof (such as indacaterol maleate). The nebulization compositions described herein may comprise an aqueous solution or suspension comprising tiotropium, or a pharmaceutically acceptable salt thereof (such as tiotropium bromide) and indacaterol, or a pharmaceutically acceptable salt thereof (such as indacaterol maleate), wherein the composition is free of any other pharmaceutically acceptable excipients.

The nebulization compositions described herein may include one or more pharmaceutically acceptable excipients. Such compositions can be contained in a single chamber container. Such a single composition comprising both tiotropium, or a pharmaceutically acceptable salt thereof (such as tiotropium bromide) and indacaterol, or a pharmaceutically acceptable salt thereof (such as indacaterol maleate) is stable during long term storage.

Alternatively, the two active agents may be formulated separately and contained in separate containers, which are then mixed prior to administration to a patient. In this instance, a composition comprising tiotropium, or a pharmaceutically acceptable salt thereof (such as tiotropium bromide), optionally with one or more pharmaceutically acceptable excipients is contained in one chamber and another composition comprising indacaterol, or a pharmaceutically acceptable salt thereof (such as indacaterol maleate), optionally with one or more pharmaceutically acceptable excipients is contained in a separate chamber. This combination comprising separate compositions of tiotropium, or a pharmaceutically acceptable salt thereof (such as tiotropium bromide) and indacaterol, or a pharmaceutically acceptable salt thereof (such as indacaterol maleate) can be contained in a dual chamber container. The two chambers of the dual chamber container may be separate or may be connected by a common septum. The dual chamber container with a common septum may contain a common top which when broken would provide simultaneous access to both compositions. The composition comprising tiotropium, or a pharmaceutically acceptable salt thereof (such as tiotropium bromide) may be mixed with the composition comprising indacaterol, or a pharmaceutically acceptable salt thereof (such as indacaterol maleate) before being added to the reservoir of the nebulizer. In one instance, the composition comprising tiotropium, or a pharmaceutically acceptable salt thereof (such as tiotropium bromide) may be added to the reservoir of the nebulizer followed by addition of the composition comprising indacaterol, or a pharmaceutically acceptable salt thereof (such as indacaterol maleate) and vice versa. In this instance, the two compositions would be mixed in the reservoir of the nebulizer device and then administered to the patient.

The nebulization compositions described herein may contain about 1 μg/ml to about 200 μg/ml, such as from about 1 μg/ml to about 5 μg/ml, from about 5 μg/ml to about 10 μg/ml, from about 10 μg/ml to about 15 μg/ml, from about 15 μg/ml to about 20 μg/ml, from about 20 μg/ml to about 40 μg/ml, from about 40 μg/ml to about 80 μg/ml, from about 80 μg/ml to about 100 μg/ml, or from about 100 μg/ml to about 200 μg/ml of tiotropium, or a pharmaceutically acceptable salt thereof, e.g., tiotropium bromide.

The nebulization compositions described herein may contain about 1 μg to about 200 μg, such as from about 1 μg to about 5 μg, from about 5 μg to about 10 μg, from about 10 μg to about 15 μg, from about 15 μg to about 20 μg, from about 20 μg to about 40 μg, from about 40 μg to about 80 μg, from about 80 μg to about 100 μg, from about 100 μg to about 200 μg of tiotropium or a pharmaceutically acceptable salt thereof, e.g., tiotropium bromide. Preferably, the nebulization compositions have a volume of 2 ml.

The nebulization compositions described herein may contain tiotropium, or a pharmaceutically acceptable salt thereof, e.g., tiotropium bromide, in un-micronized form. In an embodiment, the nebulization compositions described herein contain crystalline tiotropium bromide monohydrate in an un-micronized form. The nebulization compositions described herein may contain tiotropium, or a pharmaceutically acceptable salt thereof, e.g., tiotropium bromide, in micronized form. Suitable micronization techniques, including microfluidizer, high pressure homogenizer, ball mill, sonication and other such techniques commonly known in the art can be employed to effectively size reduce the size of the tiotropium, or its pharmaceutically acceptable salt such as tiotropium bromide. The particle size of tiotropium or its pharmaceutically acceptable salt desired for effective nebulization of the compositions of the present invention can range from about 0.1 micron to about 5 micron. In one embodiment, the nebulization compositions described herein contain tiotropium or its pharmaceutically acceptable salt having a D₉₀ of not more than about 10 microns.

The nebulization compositions described herein may contain about 1 μg/ml to about 500 μg/ml, such as from about 1 μg/ml to about 10 μg/ml, from about 10 μg/ml to about 25 μg/ml, from about 25 μg/ml to about 75 μg/ml, from about 75 μg/ml to about 100 μg/ml, from about 100 μg/ml to about 150 μg/ml, from about 150 μg/ml to about 300 μg/ml, from about 300 μg/ml to about 500 μg/ml of indacaterol, or a pharmaceutically acceptable salt thereof, e.g., indacaterol maleate.

The nebulization compositions described herein may contain about 1 μg to about 500 μg, such as from about 1 μg to about 10 μg, such as from about 10 μg to about 25 μg, from about 25 μg to about 75 μg, from about 75 μg to about 100 μg, from about 100 μg to about 150 μg, from about 150 μg to about 300 μg, from about 300 μg to about 500 μg of indacaterol, or a pharmaceutically acceptable salt thereof, e.g., indacaterol maleate. Preferably, the nebulization compositions have a volume of 2 ml.

The nebulization compositions described herein may contain indacaterol, or a pharmaceutically acceptable salt thereof, e.g., indacaterol maleate, in micronized form. In another embodiment, indacaterol may be present in an un-micronized form. Suitable micronization techniques, including microfluidizer, high pressure homogenizer, ball mill, sonication and other such techniques commonly known in the art can be employed to effectively size reduce the size of the indacaterol, or its pharmaceutically acceptable salt such as indacaterol maleate. The particle size of indacaterol or its pharmaceutically acceptable salt desired for effective nebulization of the compositions of the present invention can range from about 0.1 micron to about 5 micron. In one embodiment, the nebulization compositions described herein contain indacaterol or its pharmaceutically acceptable salt having a D₉₀ of not more than about 10 microns.

The weight ratio of tiotropium, or a pharmaceutically acceptable salt thereof (such as tiotropium bromide), and indacaterol, or a pharmaceutically acceptable salt thereof (such as indacaterol maleate) in the nebulization compositions described herein may range from tiotropium:indacaterol of from about 1:0.1 to about 1:500, such as from about 1:0.2 to about 1:100.

Suitable pharmaceutically acceptable excipients for use in any of the nebulizable compositions described herein include, but are not limited to, pH adjusting agents, isotonicity agents, chelating agents, surfactants, anti-oxidants, and pharmaceutically acceptable vehicles. In one embodiment, any of the nebulizable compositions described herein include a pH adjusting agent, an isotonicity agent, a pharmaceutically acceptable vehicle and, optionally, a chelating agent.

The nebulization compositions described herein may be substantially free of preservative (such as benzalkonium and salts thereof), preferably substantially free of benzalkonium chloride. The term “substantially free of preservative” means that the amount of preservative is not an amount sufficient to materially act as a preservative for the nebulization composition. In one embodiment, the preservative may be present in a concentration less than about 0.008% w/w based on total weight of the composition. A composition is “substantially benzalkonium chloride free” or “substantially free of benzalkonium chloride” when the amount of benzalkonium chloride is not an amount sufficient to materially act as a preservative for the nebulization composition. In one embodiment, benzalkonium chloride may be present in a concentration less than about 0.008% w/w based on the total weight of the composition.

The nebulization compositions may contain benzalkonium chloride in an amount of about 0.002% w/v to about 0.02% w/v of the composition.

Generally, nebulization compositions contain a preservative such as benzalkonium chloride. A common problem with benzalkonium chloride is that it may cause paradoxic bronchoconstriction if the solution is administered repeatedly over short intervals, and frequent exposure to benzalkonium chloride may lead to occupational asthma. Another problem is that when inhaled by patients, the benzalkonium chloride can cause dose-dependent bronchoconstriction. The nebulization compositions described herein may be provided without benzalkonium chloride, thereby making them suitable for repeated administration over a short period of time. Also, administering a substantially benzalkonium chloride free nebulization composition to a patient reduces the concomitant liability of adverse effects associated with benzalkonium chloride alone or in combination with other excipients and/or the active agents. It also negates the toxicity and other side effects associated with benzalkonium chloride.

The nebulization compositions described herein may be free, or substantially free, of complexing agents, such as ethylene diamine tetra-acetic acid (EDTA) and salts thereof. The absence of or reduction in the concentration of the additive EDTA and its salts helps to reduce the paradoxic effects associated with cough. In one embodiment of any of the nebulization compositions described herein, the composition includes no more than 0.02, 0.01, or 0.005% w/v of complexing agent, such as edetate disodium.

In one embodiment, the nebulization compositions described herein may contain from about 0.01% w/v to about 0.05% w/v of edetate disodium.

The pH of the nebulization compositions described herein may vary from about 2 to about 8. The pH may be adjusted by the addition of one or more pharmaceutically acceptable acids. Non-limiting examples of suitable pharmaceutically acceptable acids include inorganic acids, such as hydrochloric acid, hydrobromic acid, nitric acid, sulfuric acid, and phosphoric acid, and any combination of the foregoing. Non-limiting examples of other suitable pharmacologically acceptable acids include organic acids, such as ascorbic acid, citric acid, malic acid, maleic acid, tartaric acid, succinic acid, fumaric acid, acetic acid, formic acid, propionic acid, and any combination of the foregoing. The pH adjusting agents may be selected from one or more organic acids selected from ascorbic acid, fumaric acid and citric acid. A preferred organic acid is citric acid. Mixtures of the abovementioned acids may also be used, particularly in the case of acids which have other properties in addition to their acidifying properties, e.g., those which act as flavorings or antioxidants, such as citric acid and ascorbic acid.

Any of the nebulization compositions described herein may optionally include a buffer. Suitable general and biological buffers that may be used, such as those in the pH range of about 2 to about 8 include, but are not limited to, acetate, barbital, borate, Britton-Robinson, cacodylate, citrate, collidine, formate, maleate, Mcllvaine, phosphate, Prideaux-Ward, phosphate, citrate, borate, succinate, citrate-phosphate-borate (Teorell-Stanhagen), veronal acetate, 2-(N-morpholino)ethanesulfonic acid (MES), BIS-TRIS, N-(2-Acetamido)iminodiacetic acid, N-(2-Acetamido)-2-aminoethanesulfonic acid (ADA), piperazine-N,N′-bis(2-ethanesulfonic acid) (PIPES), β-Hydroxy-4-morpholinepropane sulfonic acid (MOPSO), 1,3-bis(tris(hydroxymethyl)methylamino)propane (BIS TRIS Propane), N,N-Bis(2-hydroxyethyl)-2-aminoethanesulfonic acid (BES), (3-(N-morpholino)propanesulfonic acid) (MOPS), N-[Tris(hydroxymethyl)methyl]-2-aminoethanesulfonic acid (TES), (4-(2-hydroxyethyl)-1-piperazineethanesulfonic acid) (HEPES), N,N-Bis(2-hydroxyethyl)-3 -amino-2-hydroxypropanesulfonic acid (DIPSO), (3-(N-morpholino)propanesulfonic acid) (MOBS), 2-Hydroxy-3-[tris(hydroxymethyl)methylamino]-1-propane sulfonicacid (TAPSO), [Tris(hydroxymethyl)aminomethane](TRIZMA), (2-Hydroxyethyl)-piperazine-N-2-hydroxypropanesulfonic acid) (HEPPSO), Piperazine-1,4-bis(2-hydroxypropanesulfonic acid) dihydrate (POPSO), Triethanolamine (TEA), 4-(2-Hydroxyethyl)-1-piperazinepropanesulfonic acid (EPPS), (N-Tris(Hydroxymethyl) Methylglycine) (TRICINE), Diglycine (GLY-GLY), N,N-Bis(2-hydroxyethyl)glycine (BICINE), N-(2-Hydroxyethyl)piperazine-N′-(4-butanesulfonic acid) (HEPBS), N-[Tris(hydroxymethyl)methyl]-3-aminopropanesulfonic acid (TAPS), and 2-Amino-2-methyl-1,3-propanediol (AMPD) buffers. In certain embodiments, the buffer is sodium hydrogen phosphate dihydrate, anhydrous disodium hydrogen phosphate buffer, citric acid monohydrate, trisodium citrate dihydrate, or any combination thereof.

The nebulization compositions described herein may contain from about 0.5% w/v to about 1.5% w/v of sodium dihydrogen phosphate dihydrate, from about 0.05% w/v to about 0.5% w/v of anhydrous disodium phosphate hydrogen phosphate, from about 0.05% w/v to about 2% w/v citric acid monohydrate, from about 0.05% w/v to about 2% w/v citric acid anhydrous, from about 0.1% w/v to about 1.83% w/v trisodium citrate dihydrate, from about 0.1% w/v to about 1.83% w/v Monosodium citrate, from about 0.05% w/v to about 1% w/v tartaric acid, from about 0.05% w/v to about 1% w/v phosphoric acid, or from about 0.5% w/v to about 1.5% w/v sodium tartrate dihydrate or any combination of the foregoing.

The osmolality of the nebulization compositions described herein may be from about 200 to about 500 mOsm/kg. The nebulization compositions described herein may comprise a tonicity adjusting agent, such as an ionic salt (e.g., about 0.0001% w/v to about 264% w/v ionic salt). Suitable tonicity adjusting agents include, but are not limited to, ammonium carbonate, ammonium chloride, ammonium lactate, ammonium nitrate, ammonium phosphate, ammonium sulfate, ascorbic acid, bismuth sodium tartrate, boric acid, calcium chloride, calcium disodium edetate, calcium gluconate, calcium lactate, citric acid, dextrose, diethanolamine, dimethyl sulfoxide, edetate disodium, edetate trisodium monohydrate, fluorescein sodium, fructose, galactose, glycerin, lactic acid, lactose, magnesium chloride, magnesium sulfate, mannitol, polyethylene glycol, potassium acetate, potassium chlorate, potassium chloride, potassium iodide, potassium nitrate, potassium phosphate, potassium sulfate, propylene glycol, silver nitrate, sodium acetate, sodium bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, zinc sulfate, and any combination of any of the foregoing.

The nebulization compositions described herein may also contain an osmotic adjusting agent. Suitable osmotic adjusting agents include, but are not limited to, sodium chloride, potassium chloride, zinc chloride, calcium chloride and any combination of any of the foregoing. Other osmotic adjusting agents include, but are not limited to, mannitol, glycerol, dextrose and any combination of any of the foregoing.

The nebulization compositions described herein may contain about 0.25% to about 1.13% w/v of sodium chloride.

The pharmaceutically acceptable vehicle in the nebulization compositions described herein includes water and optionally a co-solvent. Any co-solvent that is suitable for inhalation and capable of dissolving or solubilizing the tiotropium (or pharmaceutically acceptable salt thereof) in the mixture of co-solvent and water can be used. Examples of suitable co-solvents include, for example, alcohols, ethers, hydrocarbons, and perfluorocarbons. Preferably, the co-solvent is a short chain polar alcohol. More preferably, the co-solvent is an aliphatic alcohol having from one to six carbon atoms, such as ethanol or isopropanol. A preferred co-solvent is ethanol. Non-limiting examples of suitable hydrocarbons include n-butane, isobutane, pentane, neopentane and isopentanes. Non-limiting examples of suitable ethers include dimethyl ether and diethyl ether. Non-limiting examples of suitable perfluorocarbons include perfluoropropane, perfluorobutane, perfluorocyclobutane, and perfluoropentane.

Suitable surfactants for use in the nebulization compositions described herein include, but are not limited to, C₅₋₂₀-fatty alcohols, C₅₋₂₀-fatty acids, C₅₋₂₀-fatty acid esters, lecithin, glycerides, propylene glycol esters, polyoxyethylenes, polysorbates, sorbitan esters, carbohydrates, and any combination of any of the foregoing. C₅₋₂₀-fatty acids, propylene glycol diesters of the C₅₋₂₀-fatty acids, triglycerides of the C₅₋₂₀-fatty acids, and sorbitans of the C₅₋₂₀-fatty acids are preferred. Preferably, the surfactant is selected from oleic acid, sorbitan mono-, di- or tri-oleates, and any combination of any of the foregoing.

The nebulization compositions described herein may contain about 0.01% w/v to about 0.4% w/v polysorbate 80 or from about 0.01% w/v to about 0.4% w/v polysorbate 20 or from about 0.001% w/v to about 0.4% w/v sorbitan monolaurate.

Suitable antioxidants for use in the nebulization compositions described herein include, but are not limited to, ascorbic acid, vitamin A, vitamin E, tocopherols, and any combination of any of the foregoing.

The nebulization compositions described herein may also contain a complexing agent. Suitable examples of complexing agents include, but are not limited to, EDTA and salts thereof, such as edetate disodium. In one embodiment, the nebulization composition compositions described herein contain about 0.0001% to about 0.75% w/v edetate disodium, such as about 0.01% to about 0.05% w/v edetate disodium.

The nebulization compositions described herein may have a fill volume of from about 0.1 ml to about 6 ml.

The nebulization compositions described herein may be contained in a unit-dose, low-density polyethylene (LDPE) container. Each unit-dose container may be disposed in a foil pouch, and each foil pouch may contain one or more unit-dose containers. Each foil pouch containing the unit dose container may be disposed in a shelf carton. The container may have a TWIST-FLEX™ top, such top comprising an easy-to-grip tab-like handle such that the container may be opened, for example, by twisting off the tab by hand. The TWIST-FLEX™ top is advantageous in that it allows for easy dispensing of the solution, prevents spillage and eliminates the need to open the container by cutting or tearing off the top, thereby reducing cross-contamination. One or more of the single unit dose containers may be prepackaged in an aluminum foil pouch, such that the foil provides a protective barrier against environmental contaminants and light as it helps to improve the shelf-life and stability of the nebulization composition. Dispensing vials may include, but are not limited to, any container comprising glass, low density polyethylene, or any other material capable of preventing the solution from leaking out of the container. The vial may be enclosed by any conventional means including, but not limited to, screw cap, heat seal, snap-on top, flip-top, twist-off stopper, and peel away top.

The nebulization compositions described herein may be a prepackaged, sterile, premixed, premeasured nebulization composition comprising tiotropium, or a pharmaceutically acceptable salt thereof (such as tiotropium bromide) and indacaterol, or a pharmaceutically acceptable salt thereof (such as indacaterol maleate). Preferably, the nebulization composition is a ready-to-use dosage form which does not require any mixing or dilution by the subject prior to administration. The nebulization composition may be administered for the relief of bronchospasm in a subject suffering from COPD or asthma.

The nebulization compositions described herein can comprise a prefilled container containing a nebulization composition comprising tiotropium, or a pharmaceutically acceptable salt thereof (such as tiotropium bromide) and indacaterol, or a pharmaceutically acceptable salt thereof (such as indacaterol maleate), and optionally one or more pharmaceutically acceptable excipients, wherein the nebulization composition is in a solid dosage form, e.g. powder form which can be reconstituted prior to nebulization, with an appropriate diluent supplied in another prefilled container. The solid dosage form can be prepared by various methods, such as dry mixing, spray drying, lyophilization and the like.

In one embodiment, one or more prefilled containers containing a nebulization composition as described herein is provided. Each container comprises a single unit dose of a nebulization composition comprising tiotropium, or a pharmaceutically acceptable salt thereof (such as tiotropium bromide) and indacaterol, or a pharmaceutically acceptable salt thereof (such as indacaterol maleate), for the treatment of COPD or asthma. Each such container includes a sterile, premixed, premeasured, aqueous solution or suspension comprising a single unit dose of a therapeutically effective amount of tiotropium, or a pharmaceutically acceptable salt thereof (such as tiotropium bromide) and indacaterol, or a pharmaceutically acceptable salt thereof (such as indacaterol maleate) in a single container.

In one embodiment, the nebulization composition is a prefilled container containing about 2 ml of a nebulization composition comprising about 1 μg to about 200 μg of tiotropium bromide equivalent to tiotropium base and about 1 μg to about 500 μg indacaterol maleate equivalent to indacaterol base. The nebulization composition may additionally contain a surfactant, a buffer, an isotonicity agent and, optionally, a complexing agent.

In one embodiment, the nebulization composition is a prefilled container containing about 2 ml of a nebulization composition comprising about 1 μg to about 200 μg of tiotropium bromide equivalent to tiotropium base and about 1 μg to about 500 μg indacaterol maleate equivalent to indacaterol base, wherein the composition further comprises a complexing agent selected from edetate disodium and an isotonicity agent selected from sodium chloride.

In one embodiment, the nebulization composition is a prefilled container containing about 2 ml of a nebulization composition comprising about 1 μg to about 200 μg of tiotropium bromide equivalent to tiotropium base and about 1 μg to about 500 μg indacaterol maleate equivalent to indacaterol base, wherein the composition further comprises a buffering agent selected from citric acid monohydrate and trisodium citrate dihydrate, and an isotonicity agent selected from sodium chloride.

In one embodiment, the nebulization composition is a prefilled container containing about 2 ml of a nebulization composition comprising about 1 μg to about 200 μg of tiotropium bromide equivalent to tiotropium base and about 1 μg to about 500 μg indacaterol maleate equivalent to indacaterol base, wherein the composition further comprises a buffering agent selected from citric acid monohydrate and trisodium citrate dihydrate, a complexing agent selected from edetate disodium, and an isotonicity agent selected from sodium chloride.

In one embodiment, the nebulization composition is a prefilled container containing about 2 ml of a nebulization composition comprising about 1 μg to about 200 μg of tiotropium bromide equivalent to tiotropium base and about 1 μg to about 500 μg indacaterol maleate equivalent to indacaterol base, wherein the composition further comprises a buffering agent selected from citric acid monohydrate and trisodium citrate dihydrate, a surfactant selected from Polysorbate 20 and Polysorbate 80, and an isotonicity agent selected from sodium chloride.

In one embodiment, the nebulization composition is a prefilled container containing about 2 ml of the nebulization composition comprising about 1 μg to about 200 μg of tiotropium bromide equivalent to tiotropium base and about 1 μg to about 500 μg indacaterol maleate equivalent to indacaterol base, wherein the composition further comprises a buffering agent selected from sodium dihydrogen phosphate dihydrate and anhydrous disodium hydrogen phosphate, a complexing agent selected from edetate disodium, and an isotonicity agent selected from sodium chloride.

In one embodiment, the nebulization composition is a prefilled container containing about 2 ml of the nebulization composition comprising about 1 μg to about 200 μg of tiotropium bromide equivalent to tiotropium base and about 1 μg to about 500 μg indacaterol maleate equivalent to indacaterol base, wherein the composition further comprises a buffering agent selected from sodium dihydrogen phosphate dihydrate and anhydrous disodium hydrogen phosphate, and an isotonicity agent selected from sodium chloride.

In another embodiment, a sterile pharmaceutical composition is a multi-dose nebulizable composition comprising tiotropium, or a pharmaceutically acceptable salt thereof and indacaterol, or a pharmaceutically acceptable salt thereof. The sterile pharmaceutical composition may be administered in nebulized form to relieve bronchospasm in a subject, such as a subject suffering from COPD or asthma.

In one embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and monosodium citrate, a complexing agent selected from edetate disodium, and a preservative selected from benzalkonium chloride.

In another embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and monosodium citrate, and a preservative selected from benzalkonium chloride.

In one more embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and monosodium citrate, and a complexing agent selected from edetate disodium.

In a further embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and a buffering agent selected from tartaric acid and monosodium citrate.

In one embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and sodium tartrate dihydrate, a complexing agent selected from edetate disodium, and a preservative selected from benzalkonium chloride.

In another embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and sodium tartrate dihydrate, and a preservative selected from benzalkonium chloride.

In one more embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from tartaric acid and sodium tartrate dihydrate, and a complexing agent selected from edetate disodium.

In a further embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and a buffering agent selected from tartaric acid and sodium tartrate dihydrate.

In one embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, a complexing agent selected from edetate disodium, and a preservative selected from benzalkonium chloride.

In another embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, and a preservative selected from benzalkonium chloride.

In one more embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a buffering agent selected from phosphoric acid and sodium dihydrogen phosphate dihydrate, and a complexing agent selected from edetate disodium.

In a further embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises a isotonicity agent selected from sodium chloride, and a buffering agent selected from phosphoric acid and sodium dihydrogen phosphate dihydrate.

In one embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, a complexing agent selected from edetate disodium, a preservative selected from benzalkonium chloride and an acid as pH adjusting agent.

In another embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, an acid as pH adjusting agent, and a preservative selected from benzalkonium chloride.

In one more embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, an acid as pH adjusting agent, and a complexing agent selected from edetate disodium.

In a further embodiment, the nebulization composition is a prefilled container containing a nebulization composition comprising about 1 μg to about 200 μg of tiotropium bromide equivalent to tiotropium base and about 1 μg to about 500 μg indacaterol maleate equivalent to indacaterol base, wherein the composition further comprises an isotonicity agent selected from sodium chloride, and an acid as pH adjusting agent.

In one embodiment, the nebulization compositions as described herein contain not more than about 0.7% of tiotropium Impurity A.

In another embodiment, the nebulization compositions as described herein contain not more than about 0.7% total impurity for tiotropium.

In one more embodiment, the nebulization composition as described herein contain not more than about 0.1% monoethyl impurity of indacaterol (i.e., 5-((1R)-2-((5-ethyl-2,3-dihydro-1H-inden-2-yl)amino)-1-hydroxyethyl)-8-hydroxyquinolin-2(1H)-one).

In an embodiment, the nebulization composition as described herein contain not more than about 2% total impurity for indacaterol.

The nebulization compositions described herein have a long shelf life, i.e., they are stable during long term storage. The nebulization compositions described herein may contain greater than about 80%, such as greater than about 85%, greater than about 90%, greater than about 95% or greater than about 98% of the initial amount of tiotropium (or its salt) and indacaterol (or its salt) after being stored for 3 or 6 months or 1, 2 or 3 years at 2-8° C. or 25° C. when stored in a suitable container (such as a LDPE container in an aluminum pouch). The stability may be determined using Arrhenius kinetics.

The nebulization composition described herein exhibit better physical stability and chemical stability than typical formulations.

The nebulization compositions described herein may be administered by a suitable nebulizer. Suitable nebulizers include, but are not limited to, a jet nebulizer, an ultrasonic nebulizer, vibrating mesh nebulizer and a breath actuated nebulizer. Preferably, the nebulizer is a jet nebulizer connected to an air compressor with adequate airflow. The nebulizer being equipped with a mouthpiece or suitable face mask. Exemplary jet nebulizers for use herein include Pari LCplus/ProNeb, Pari LC plus/ProNeb Turbo, Pari LC plus/Dura Neb 1000 & 2000, Pari LC plus/Walkhaler, Pari LC plus/Pari Master, Pari LC star, Omron CompAir XL Portable Nebulizer System (NE-C 18 and JetAir Disposable nebulizer), Omron CompAir Elite Compressor Nebulizer System (NE-C21 and Elite Air Reusable Nebulizer), Pari LC Plus or Pari LC Star nebulizer with Proneb Ultra compressor, Pulmo-aide, Pulmo-aide LT, Pulmo-aide traveler, Invacare Passport, Inspiration Healthdyne 626, Pulmo-Neb Traverler, DeVilbiss 646, Whisper Jet, Acorn II, Misty-Neb, Allied aerosol, Schuco Home Care, Lexan Plasic Pocet Neb, SideStream Hand Held Neb, Mobil Mist, Up-Draft, Up-Draft II, T Up-Draft, ISO-NEB, AVA-NEB, Micro Mist, and PulmoMate. Exemplary ultrasonic nebulizers for use herein include MicroAir, UltraAir, Siemens Ultra Nebulizer 145, CompAir, Pulmosonic, Scout, 5003 Ultrasonic Neb, 5110 Ultrasonic Neb, 5004 Desk Ultrasonic Nebulizer, Mystique Ultrasonic, Luminscope's Ultrasonic Nebulizer, Medisana Ultrasonic Nebulizer, Microstat Ultrasonic Nebulizer, and MABISMist Hand Held Ultrasonic Nebulizer. Other nebulizers for use herein include 5000 Electromagnetic Neb, 5001 Electromagnetic Neb 5002 Rotary Piston Neb, Lumineb I Piston Nebulizer 5500, AERONEB™ Portable Nebulizer System, AERODOSE™ Inhaler, AeroEclipse Breath Actuated Nebulizer, HALOLITE™ system (Profile Therapeutics), AKITA® systems (InaMed, Germany), Mystic system (BattellePharma), RESPIMAT® (Boehringer Ingelheim), Microbase®, AERX® (Aradigm), and E-FLOW™ (Pari). Additionally, the formulations described herein can also be nebulized using inhalers other than those described above, for example jet-stream inhalers or by breath actuated jet nebulizers.

In one embodiment, any of the nebulization compositions described herein, when administered by a nebulizer provides a mass median aerodynamic diameter (MMAD) of below about 10 microns, such as between about 3 and about 6 microns.

In another embodiment, any of the nebulization compositions described herein, when administered by a nebulizer provides a geometric standard deviation (GSD) of below about 5, such as between about 1 and about 3.

In another embodiment, any of the nebulization compositions described herein, when administered by a nebulizer device provides a fine particle dose which is not less than about 10%.

In another embodiment of any of the nebulization compositions described herein, the fine particle fraction (FPF) obtained following administration of the nebulizable composition in a nebulizer is about 10% to about 70%.

In another embodiment of any of the nebulization compositions described herein, the time taken to nebulize the nebulizable composition from the nebulizer device is about 1 to about 10 minutes.

In another embodiment of any of the nebulization compositions described herein, the nebulization composition exhibits a delivered dose between about 40% to about 90%.

In one embodiment, the nebulization compositions described herein can be prepared as follows:

(i) Dissolving pharmaceutically acceptable excipients such as isotonicity agents, buffers and/or pH adjustors and optionally complexing agents and surfactants in water for injection;

(ii) Adding indacaterol or a pharmaceutically acceptable salt thereof followed by optional sonication;

(iii) Adding tiotropium or a pharmaceutically acceptable salt thereof;

(iv) Filtering the solution of step (iii) under aseptic conditions; and

(v) Filling the solution of step (iv) into a low-density polyethylene (LDPE) container.

In another aspect, the present invention relates to a method of administering tiotropium, or a pharmaceutically acceptable salt thereof (such as tiotropium bromide), and indacaterol, or a pharmaceutically acceptable salt thereof (such as indacaterol maleate), the method comprising administering by inhalation to a subject in need thereof a nebulization composition according to any of the embodiments described herein. The nebulization compositions comprising tiotropium, or a pharmaceutically acceptable salt thereof (such as tiotropium bromide), and indacaterol, or a pharmaceutically acceptable salt thereof (such as indacaterol maleate) can be administered to a subject at least once daily, or twice daily.

In another aspect, the present invention also relates to a method of relieving bronchospasm (such as bronchospasm associated with COPD or asthma), the method comprising administering by inhalation to a subject in need thereof a nebulization composition according to any of the embodiments described herein. The nebulization compositions comprising tiotropium, or a pharmaceutically acceptable salt thereof (such as tiotropium bromide), and indacaterol, or a pharmaceutically acceptable salt thereof (such as indacaterol maleate) can be administered to a subject at least once daily, or twice daily. The nebulization compositions described herein provide a faster onset of relief from bronchospasms (such as that associated with COPD or asthma) compared to typical formulations.

In another aspect, the present invention also relates to a method of increasing the forced expiratory volume (FEV1) values in a patient by administering to the patient a nebulization composition according to any of the embodiments described herein.

The nebulization compositions described herein can be supplied as a part of a kit and/or system for administering a bronchodilator to relieve bronchospasm, for instance, bronchospasm associated with COPD or asthma. The kit and/or system may comprise a nebulization composition according to any of the embodiments described herein. In one instance, the kit and/or system comprises a nebulization composition comprising a therapeutically effective amount of tiotropium, or a pharmaceutically acceptable salt thereof (such as tiotropium bromide), and indacaterol, or a pharmaceutically acceptable salt thereof (such as indacaterol maleate) in a prepackaged, premeasured, premixed and/or single unit dose form for the treatment of COPD or asthma. In another instance, the prepackaged inhalation kit and/or system comprises one or more premixed, premeasured single unit dose vials comprising a nebulization composition comprising a therapeutically effective amount of tiotropium, or a pharmaceutically acceptable salt thereof (such as tiotropium bromide), and indacaterol, or a pharmaceutically acceptable salt thereof (such as indacaterol maleate) for the treatment of bronchospasm (such as that associated with COPD or asthma), and instructions for using the same.

The nebulization compositions described herein can also be supplied as a part of a kit, the kit comprising a nebulizer, instructions for using the nebulizer and the unit dose vial(s) containing the nebulization compositions described herein. The time taken for administering the nebulization composition may be from about 1 minute to about 10 minutes. The nebulization compositions comprising tiotropium, or a pharmaceutically acceptable salt thereof (such as tiotropium bromide), and indacaterol, or a pharmaceutically acceptable salt thereof (such as indacaterol maleate) can be administered to a subject at least once daily, such as twice daily.

In another aspect, the present invention relates to a kit for the treatment, prevention or amelioration or one or more symptoms of diseases or disorders associated with bronchoconstriction which comprises:

(i) a nebulizer;

(ii) a nebulization composition (e.g., for the treatment, prevention or amelioration or one or more symptoms of diseases or disorders associated with bronchoconstriction) comprising:

(a) tiotropium, or a pharmaceutically acceptable salt thereof (such as tiotropium bromide) and indacaterol, or a pharmaceutically acceptable salt thereof (such as indacaterol maleate); and

(b) water.

In another aspect, the present invention relates to a device comprising tiotropium, or a pharmaceutically acceptable salt thereof (such as tiotropium bromide) and indacaterol, or a pharmaceutically acceptable salt thereof (such as indacaterol maleate), for use, for example, in relieving the symptoms of COPD or asthma.

In another aspect, the present invention relates to a method for improving user compliance and/or quality of life in patients with asthma or COPD, compared to conventional treatments. The method comprises initiating treatment with a nebulization composition, or a container, kit, or system according to any of the embodiments described herein.

Throughout this specification it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

The following examples further illustrates the invention but are not limiting.

### EXAMPLE 1

| | Amount in %w/v | | | | | | | |
|---|---|---|---|---|---|---|---|---|
| | # | | | | | | | |
| | 1 | 2 | 3 | 4 | 5 | 6 | 7 | 8 |
| Tiotropium | 0.0001-0.008 | | | | | | | |
| bromide | | | | | | | | |
| Indacaterol | 0.0003-0.064 | | | | | | | |
| maleate | | | | | | | | |
| Sodium | 0.9 | 0.9 | 0.9 | 0.9 | 0.9 | 0.9 | 0.9 | 0.9 |
| chloride | | | | | | | | |
| Citric acid | 1.923 | 1.923 | 0.961 | 3.845 | 1.923 | 1.923 | — | — |
| monohydrate | | | | | | | | |
| Trisodium | 0.25 | 0.25 | 0.125 | 0.5 | 0.25 | 0.25 | — | — |
| citrate | | | | | | | | |
| dihydrate | | | | | | | | |
| Edetate | 0.01-0.02 | — | — | — | — | — | 0.01-0.02 | 0.01-0.02 |
| disodium | | | | | | | | |
| HCl | — | — | — | — | — | — | — | q.s. |
| Polysorbate 20 | — | — | — | — | 0.02 | — | — | — |
| Polysorbate 80 | — | — | — | — | — | 0.02 | — | — |
| Sodium | — | — | — | — | — | — | 0.94 | — |
| dihydrogen | | | | | | | | |
| phosphate | | | | | | | | |
| dihydrate | | | | | | | | |
| Anhydrous | — | — | — | — | — | — | 0.175 | — |
| disodium | | | | | | | | |
| hydrogen | | | | | | | | |
| phosphate | | | | | | | | |
| Water for | q.s. | q.s. | q.s. | q.s. | q.s. | q.s. | q.s. | q.s. |
| injection | | | | | | | | |

### Manufacturing Process

1. Dissolve sodium chloride, edetate disodium, citric acid monohydrate, trisodium citrate dihydrate or sodium dihydrogen phosphate dihydrate, and anhydrous disodium hydrogen phosphate in water for injection with stirring. Add hydrochloric acid to adjust the pH (composition #8).
2. Check the clarity of the solution.
3. Add indacaterol maleate (optionally dissolved in Polysorbate 20 or Polysorbate 80) to the product of step 1 with stirring.
4. Sonicate until a clear solution is obtained.
5. Add tiotropium bromide to the solution of step 4 with stirring. Make up the volume with water for injection.
6. Filter the product of step 5 through a sterilizing grade filter.
7. Fill LDPE vials with the product of Step 6.

### EXAMPLE 2

| | Amount in μg/2 ml | | | |
|---|---|---|---|---|
| # | 1 | 2 | 3 | 4 |
| Tiotropium | 5-40 μg/2 ml | | | |
| bromide | | | | |
| Indacaterol | 10-300 μg/2 ml | | | |
| maleate | | | | |
| Sodium chloride | 16000 | 16000 | 16000 | 16000 |
| Tartaric acid | 5000 | 5000 | 5000 | 5000 |
| Monosodium Citrate | 2000 | 2000 | 2000 | 2000 |
| Edetate disodium | 200 | — | 200 | — |
| Benzalkonium Chloride | 400 | 400 | — | — |
| Water for injection | q.s. 2 ml | q.s. 2 ml | q.s. 2 ml | q.s. 2 ml |

### Manufacturing Process

1. Dissolve batch quantities of sodium chloride, edetate disodium, tartaric acid and monosodium citrate in water for injection with stirring. Check the clarity of the solution.
2. Add indacaterol maleate to the product of Step 1.
3. Stir/homogenize/sonicate until a clear solution is obtained.
4. Add tiotropium bromide to the product of Step 3 with stirring.
5. Add benzalkonium chloride (BKC) to the product of Step 4 and mix. Make up the volume with water for injection.
6. Filter the product of Step 5 through a sterilizing grade filter.
7. Fill the product of Step 6 in LDPE smartules/HDPE, PP, LDPE bottles/amber glass bottles/amber glass vials in single dose or multidose containers

### EXAMPLE 3

| | Amount in μg/2 ml | | | |
|---|---|---|---|---|
| # | 1 | 2 | 3 | 4 |
| Tiotropium | 5-40 μg/2 ml | | | |
| bromide | | | | |
| Indacaterol | 10- 300 μg/2 ml | | | |
| maleate | | | | |
| Sodium chloride | 16000 | 16000 | 16000 | 16000 |
| Tartaric acid | 5000 | 5000 | 5000 | 5000 |
| Sodium tartrate | 2000 | 2000 | 2000 | 2000 |
| dihydrate | | | | |
| Edetate disodium | 200 | — | 200 | — |
| Benzalkonium chloride | 400 | 400 | — | — |
| Water for injection | q.s. 2 ml | q.s. 2 ml | q.s. 2 ml | q.s. 2 ml |

### Manufacturing Process

1. Dissolve batch quantities of sodium chloride, edetate disodium, tartaric acid and sodium tartrate dihydrate in water for injection with stirring. Check the clarity of the solution.
2. Add indacaterol maleate to the product of Step 1.
3. Stir/homogenize/sonicate until a clear solution is obtained.
4. Add tiotropium bromide to the product of Step 3 with stirring.
5. Add benzalkonium chloride (BKC) to the product of Step 4 and mix. Make up the volume with water for injection.
6. Filter the product of Step 5 solution through sterilizing grade filter.
7. Fill the product of Step 6 in LDPE smartules/HDPE, PP, LDPE bottles/amber glass bottles/amber glass vials in single dose or multidose containers.

### EXAMPLE 4

| | Amount in μg/2 ml | | | |
|---|---|---|---|---|
| # | 1 | 2 | 3 | 4 |
| Tiotropium bromide | 5-40 μg/2 ml | | | |
| Indacaterol maleate | 10-300 μg/2 ml | | | |
| Sodium chloride | 16000 | 16000 | 16000 | 16000 |
| Phosphoric acid | 680 | 680 | 680 | 680 |
| Sodium dihydrogen | 1560 | 1560 | 1560 | 1560 |
| phosphate dihydrate | | | | |
| Edetate disodium | 200 | — | 200 | — |
| Benzalkonium Chloride | 400 | 400 | — | — |
| Water for injection | q.s. 2 ml | q.s. 2 ml | q.s. 2 ml | q.s. 2 ml |

### Manufacturing Process

1. Dissolve batch quantities of sodium chloride, edetate disodium, phosphoric acid, sodium dihydrogen phosphate dihydrate and benzalkonium chloride in water for injection with stirring. Check the clarity of the solution.
2. Add Indacaterol maleate to the product of Step 1.
3. Stir/homogenize/sonicate until a clear solution is obtained.
4. Add tiotropium bromide to the product of Step 3 with stirring.
5. Add benzalkonium chloride (BKC) to the product of Step 4 and mix. Make up the volume with water for injection.
6. Filter the product of Step 5 through a sterilizing grade filter.
8. Fill the product of Step 6 in LDPE smartules/HDPE, PP, LDPE bottles/amber glass bottles/amber glass vials in single dose or multidose containers.

### EXAMPLE 5

| | Amount in μg/2 ml | | | |
|---|---|---|---|---|
| # | 1 | 2 | 3 | 4 |
| Tiotropium bromide | 5-40 μg/2 ml | | | |
| Indacaterol maleate | 10-300 μg/2 ml | | | |
| Sodium chloride | 18000 | 18000 | 18000 | 18000 |
| Edetate disodium | 200 | — | 200 | — |
| Benzalkonium chloride | 400 | 400 | — | — |
| HCl | q.s. | q.s. | q.s. | q.s. |
| | adjust pH | adjust pH | adjust pH | adjust pH |
| Water for injection | q.s. 2 ml | q.s. 2 ml | q.s. 2 ml | q.s. 2 ml |

### Manufacturing Process

1. Dissolve batch quantities of sodium chloride in water for injection with stirring. Check the clarity of the solution. Adjust the pH of solution to pH 2.7 with hydrochloric acid.
2. Add indacaterol maleate to the product of Step 1.
3. Stir/homogenize/sonicate until a clear solution is obtained.
4. Add tiotropium bromide to the product of Step 3 with stirring.
5. Add benzalkonium chloride (BKC) to the product of Step 4 and mix. Make up the volume with water for injection.
6. Filter the product of Step 5 through a sterilizing grade filter.

### EXAMPLE 6

### Nebulization Composition Comprising Tiotropium Bromide 20 mcg and Indacaterol Maleate 75 mcg

| # | Amount in % w/v |
|---|---|
| Tiotropium bromide | 0.001 |
| Indacaterol maleate | 0.0038 |
| Benzalkonium chloride | 0.02 |
| Sodium chloride | 0.9 |
| Hydrochloric acid | q.s. |
| Sterile water for injection | 2 ml |

The stability of the above nebulization composition was tested at 25±2° C./60±5% RH (relative humidity) and at 40±2° C./75±5% RH. The results are shown in the table below.

| | | | | | | Indacaterol | | | | |
|---|---|---|---|---|---|---|---|---|---|---|
| | | Tiotropium | | | | | | Stage III | | |
| | | Tiotropium | Impurity | Single | Total | Indacaterol | Monoethyl | (Benzyl | Single | Total |
| Test | pH | Assay | A | Max | Impurity | Assay | Impurity | Indacaterol) | Max | Impurity |
| Initial | 2.69 | 96.0 | 0.34 | ND | 0.34 | 100.9 | 0.05 | ND | 0.21 | 1.21 |

| | |
|---|---|
| Pack | HDPE Bottles (60 mL Capacity) with Polypropylene Cap and Extended Polyethylene wad |

Stability condition: 40 ± 2° C./75 ± 5% RH

| | | | | | | | | | | |
|---|---|---|---|---|---|---|---|---|---|---|
| 1 M | 2.65 | 96.1 | 0.57 | ND | 0.57 | 97.9 | 0.04 | ND | 0.40 | 1.23 |

Stability condition: 25 ± 2° C./60 ± 5% RH

| | | | | | | | | | | |
|---|---|---|---|---|---|---|---|---|---|---|
| 1 M | 2.68 | 95.0 | 0.34 | ND | 0.34 | 96.6 | 0.04 | ND | 0.34 | 1.17 |

| | |
|---|---|
| Pack | Amber Glass Bottles (60 mL Capacity) with Aluminium Cap and Extended Polyethylene wad |

Stability condition: 40 ± 2° C./75 ± 5% RH

| | | | | | | | | | | |
|---|---|---|---|---|---|---|---|---|---|---|
| 1 M | 2.64 | 94.7 | 0.38 | ND | 0.38 | 96.1 | 0.05 | ND | 0.45 | 1.11 |

Stability condition: 25 ± 2° C./60 ± 5% RH

| | | | | | | | | | | |
|---|---|---|---|---|---|---|---|---|---|---|
| 1 M | 2.69 | 94.5 | 0.21 | ND | 0.21 | 95.9 | 0.05 | ND | 0.28 | 0.98 |

| # | Amount in % w/v |
|---|---|
| Tiotropium bromide | 0.001 |
| Indacaterol maleate | 0.0038 |
| Tartaric acid | 0.25 |
| Monosodium citrate | 0.1 |
| Benzalkonium chloride | 0.02 |
| Sodium chloride | 0.8 |
| Sterile water for injection | 2 ml |

The stability of the above nebulization composition was tested at 2 -8° C., 25 ±2° C./60±5% RH (relative humidity) and at 40±2° C./75 ±5% RH. The results are shown in the table below.

| | | | | | | Indacaterol | | | | |
|---|---|---|---|---|---|---|---|---|---|---|
| | | Tiotropium | | | | | | Stage III | | |
| | | Tiotropium | Impurity | Single | Total | Indacaterol | Monoethyl | (Benzyl | Single | Total |
| Test | pH | Assay | A | Max | Impurity | Assay | Impurity | Indacaterol) | Max | Impurity |
| Initial | 2.69 | 100.6 | 0.17 | ND | 0.17 | 101.5 | 0.03 | ND | 0.13 | 0.73 |

| | |
|---|---|
| Pack | HDPE Bottles (60 mL Capacity) with Polypropylene Cap and Extended Polyethylene wad |

Stability condition: 40 ± 2° C./75 ± 5% RH

| | | | | | | | | | | |
|---|---|---|---|---|---|---|---|---|---|---|
| 1 M | 2.65 | 99.0 | 0.35 | ND | 0.35 | 99.5 | 0.05 | ND | 0.17 | 1.22 |

Stability condition: 25 ± 2° C./60 ± 5% RH

| | | | | | | | | | | |
|---|---|---|---|---|---|---|---|---|---|---|
| 1 M | 2.67 | 99.1 | 0.15 | ND | 0.15 | 99.0 | 0.04 | ND | 0.14 | 0.89 |

Stability condition: 2-8° C.

| | | | | | | | | | | |
|---|---|---|---|---|---|---|---|---|---|---|
| 1 M | 2.69 | 100.4 | 0.16 | ND | 0.16 | 101.5 | 0.05 | ND | 0.13 | 0.98 |

| | |
|---|---|
| Pack | Amber Glass Bottles (60 mL Capacity) with Aluminium Cap and Extended Polyethylene wad |

Stability condition: 40 ± 2° C./75 ± 5% RH

| | | | | | | | | | | |
|---|---|---|---|---|---|---|---|---|---|---|
| 1 M | 2.64 | 98.4 | 0.34 | ND | 0.34 | 99.2 | 0.06 | ND | 0.21 | 1.36 |

Stability condition: 25 ± 2° C./60 ± 5% RH

| | | | | | | | | | | |
|---|---|---|---|---|---|---|---|---|---|---|
| 1 M | 2.67 | 98.4 | 0.17 | ND | 0.17 | 98.9 | 0.04 | ND | 0.15 | 0.87 |

Stability condition: 2-8° C.

| | | | | | | | | | | |
|---|---|---|---|---|---|---|---|---|---|---|
| 1 M | 2.68 | 98.1 | 0.11 | ND | 0.11 | 98.4 | 0.05 | ND | 0.15 | 0.81 |

### EXAMPLE 8

| | Amount in % w/v | | | |
|---|---|---|---|---|
| # | 8A | 8B | 8C | 8D |
| Tiotropium bromide | 0.001 | 0.001 | 0.001 | 0.001 |
| Indacaterol maleate | 0.00185 | 0.00375 | 0.005 | 0.0075 |
| Sodium chloride | 0.3 | 0.3 | 0.3 | 0.3 |
| Citric acid | 1.85 | 1.85 | 1.85 | 1.85 |
| monohydrate | | | | |
| Tri sodium citrate | 0.25 | 0.25 | 0.25 | 0.25 |
| dihydrate | | | | |
| EDTA | 0.01 | 0.01 | 0.01 | 0.01 |
| Sterile water for | q.s. 2 ml | q.s. 2 ml | q.s. 2 ml | q.s. 2 ml |
| injection | | | | |

The contents of the compositions described in Examples 8A-8D were poured into the reservoir of a vibrating mesh nebulizer, such as a Microbase Device. The compositions were then evaluated using a Next Generation Impactor (NGI) device. The NGI device mimics several components of the respiratory tract.

The data shown below is a representation of the in-vitro aerodynamic particle size distribution (APSD) data by NGI and the Breath simulator (BRS) data for the nebulization compositions of Examples 8A-8D. FPM refers to fine particle mass. MB refers to mass balance. MOC refers to micro-orifice collector. IP refers to induction port. S1 to S7 refer to Stage 1 through Stage 7 respectively. DD refers to drug delivery.

### In Vitro Data—APSD

| | 8A | | 8B | | 8C | | 8D | |
|---|---|---|---|---|---|---|---|---|
| | Component | | | | | | | |
| | Tiotropium | Indacaterol | Tiotropium | IndaCaterol | Tiotropium | Indacaterol | Tiotropium | IndaCaterol |
| Stage | Delivered dose (mcg) | | | | | | | |
| IP | 0.38 | 0.54 | 0.29 | 1.12 | 0.33 | 1.39 | 0.27 | 1.95 |
| S1 | 0.53 | 1.08 | 0.82 | 3.39 | 0.93 | 4.53 | 0.57 | 4.56 |
| S2 | 0.71 | 1.40 | 1.28 | 5.06 | 1.43 | 7.03 | 0.70 | 5.59 |
| S3 | 3.67 | 6.97 | 4.48 | 17.32 | 4.74 | 22.79 | 3.27 | 25.45 |
| S4 | 7.40 | 14.09 | 6.95 | 26.73 | 7.11 | 35.09 | 7.03 | 54.32 |
| S5 | 4.50 | 8.59 | 4.28 | 16.71 | 4.03 | 19.78 | 5.04 | 38.92 |
| S6 | 1.08 | 2.27 | 1.14 | 4.77 | 1.07 | 5.34 | 1.24 | 9.94 |
| S7 | 0.23 | 0.55 | 0.23 | 1.20 | 0.25 | 1.42 | 0.24 | 2.37 |
| MOC | 0.20 | 0.14 | 0.08 | 0.74 | 0.08 | 0.36 | 0.20 | 0.74 |
| Device | 0.33 | 0.56 | 0.23 | 1.48 | 0.21 | 1.89 | 0.3 | 2.68 |
| Mass | 19.03 | 36.18 | 19.78 | 78.52 | 20.19 | 99.63 | 18.87 | 146.51 |
| balance | | | | | | | | |
| Delivered | 18.7 | 35.62 | 19.55 | 77.04 | 19.98 | 97.73 | 18.57 | 143.83 |
| dose (mcg) | | | | | | | | |
| FPD | 12.381 | 23.676 | 11.636 | 46.159 | 11.448 | 56.627 | 12.68 | 97.97 |
| ISM (S2 to | 17.79 | 34.01 | 18.44 | 72.53 | 18.71 | 91.81 | 13.96 | 137.33 |
| MOC) | | | | | | | | |
| IP to S2 | 1.62 | 3.02 | 2.39 | 9.57 | 2.69 | 12.95 | 1.54 | 12.10 |
| S3 to S5 | 15.57 | 29.65 | 15.71 | 60.76 | 15.88 | 77.66 | 15.34 | 118.69 |
| S6 to MOC | 1.51 | 2.96 | 1.45 | 6.71 | 1.40 | 7.12 | 1.68 | 13.05 |
| FPF (%) | 66.207 | 66.450 | 59.521 | 59.915 | 57.327 | 57.943 | 68.972 | 68.778 |
| MMAD | 4.052 | 4.052 | 4.356 | 4.316 | 4.487 | 4.460 | 3.885 | 3.894 |
| (μm) | | | | | | | | |
| GSD | 1.631 | 1.642 | 1.733 | 1.756 | 1.737 | 1.744 | 1.673 | 1.681 |

| | | | Total | Total |
|---|---|---|---|---|
| | | | Delivered | Delivered |
| Example | Component | Time (min) | Dose (mcg) | Dose (%) |
| 8A | Tiotropium bromide | 6 min | 10.6 mcg | 53.10% |
| | Indacaterol maleate | | 18.5 mcg | 49.40% |
| 8B | Tiotropium bromide | 6 min | 9.2 mcg | 46.10% |
| | Indacaterol maleate | | 37.5 mcg | 50.0% |
| 8C | Tiotropium bromide | 7 min | 9.9 mcg | 49.60% |
| | Indacaterol maleate | | 48.7 mcg | 48.70% |
| 8D | Tiotropium bromide | 7 min | 10.2 mcg | 51.00% |
| | Indacaterol maleate | | 74.8 mcg | 49.90% |

All references cited herein are hereby incorporated by reference.

1. A nebulization composition comprising
(i) tiotropium or a pharmaceutically acceptable salt thereof;
(ii) indacaterol or a pharmaceutically acceptable salt thereof
(iii) an isotonicity agent; and
(iv) optionally one or more pharmaceutically acceptable excipients selected from the group consisting of a pH adjusting agent, buffer, complexing agent, a preservative, a surfactant, and any combination thereof.

2. The nebulization composition according to claim 1, wherein the composition is a solution.

3. The nebulization composition according to claim 1, wherein the composition is a suspension.

4. The nebulization composition according to claim 1, wherein the composition comprises indacaterol maleate.

5. The nebulization composition according to claim 1, wherein the composition comprises micronized indacaterol maleate.

6. The nebulization composition according to claim 1, wherein the composition comprises tiotropium bromide.

7. The nebulization composition according to claim 1, wherein the composition comprises between about 1 μg/ml and about 500 μg/ml of indacaterol or a pharmaceutically acceptable salt thereof.

8. The nebulization composition according to claim 1, wherein the composition comprises between about 1 μg/ml and about 200 μg/ml of tiotropium or a pharmaceutically acceptable salt thereof.

9. The nebulization composition according to claim 1, wherein the volume of the composition is between about 0.1 ml to about 6 ml.

10. The nebulization composition according to claim 1, wherein the composition is contained in a prefilled container.

11. The nebulization composition according to claim 1, wherein the composition comprises indacaterol maleate having a mean particle size of about 0.1 micron to about 5 microns.

12. The nebulization composition according to claim 1, wherein the composition has an osmolality of about 200 to about 500 mOsm/kg.

13. The nebulization composition according to claim 1, wherein the composition when administered by a nebulizer provides a mass median aerodynamic diameter of below about 10 microns.

14. The nebulization composition according to claim 1, wherein the composition when administered by a nebulizer provides a geometric standard deviation of below about 5.

15. The nebulization composition according to claim 1, wherein the composition has a geometric standard deviation of about 1 to about 3 and a mass mean aerodynamic diameter of about 3 to about 6 microns when administered by a nebulizer device.

16. The nebulization composition according to claim 1, wherein the composition is a prepackaged, sterile, premixed, premeasured unit dose composition.

17. The nebulization composition according to claim 1, wherein the composition is administered to relieve bronchospasm in a subject.

18. The nebulization composition according to claim 1, wherein the composition is administered once daily.

19. The nebulization composition according to claim 1, wherein the composition is administered twice daily.

20. The nebulization composition according to claim 1, wherein the composition comprises indacaterol maleate having a D₉₀ of not more than 10 microns.

21. The nebulization composition according to claim 1, wherein the composition further comprises a pharmaceutically acceptable surfactant.

22. The nebulization composition according to claim 1, wherein the composition further comprises a pH adjusting agent.

23. The nebulization composition according to claim 1, wherein the composition further comprises a complexing agent.

24. The nebulization composition according to claim 1, wherein the composition further comprises a pharmaceutically acceptable preservative.

25. The nebulization composition according to claim 1, wherein the composition further comprises a pharmaceutically acceptable buffer.

26. The nebulization composition according to claim 1, wherein the pH of the composition is between about 2 and about 8, such as between about 2.0 and about 4.0.

27. The nebulization composition according to claim 1, wherein the composition contains less than about 0.5% of a monoethyl impurity of indacaterol.

28. The nebulization composition according to claim 1, wherein the composition contains less than about 1% of tiotropium Impurity A.

29. The nebulization composition of claim 1, wherein the composition comprises:
(i) tiotropium or a pharmaceutically acceptable salt thereof
(ii) indacaterol or a pharmaceutically acceptable salt thereof,
(iii) an isotonicity agent,
(iv) a pH adjusting agent; and
(v) water for injection.

30. The nebulization composition of claim 1, wherein the composition comprises
(i) tiotropium or a pharmaceutically acceptable salt thereof
(ii) indacaterol or a pharmaceutically acceptable salt thereof,
(iii) an isotonicity agent,
(iv) a buffer,
(v) optionally, one or more pharmaceutically acceptable excipients selected from the group consisting of a complexing agent, a surfactant and a preservative; and
(iv) water for injection.

31. A nebulization composition comprising:
(i) tiotropium or a pharmaceutically acceptable salt thereof
(ii) indacaterol or a pharmaceutically acceptable salt thereof,
(iii) sodium chloride,
(iv) tartaric acid and sodium tartrate dihydrate,
(v) optionally, one or more pharmaceutically acceptable excipients selected from the group consisting of a complexing agent, and a preservative; and
(iv) water for injection.

32. A nebulization composition comprising:
(i) tiotropium or a pharmaceutically acceptable salt thereof
(ii) indacaterol or a pharmaceutically acceptable salt thereof,
(iii) sodium chloride,
(iv) phosphoric acid and sodium dihydrogen phosphate dihydrate
(v) optionally, one or more pharmaceutically acceptable excipients selected from the group consisting of a complexing agent, and a preservative; and
(iv) water for injection.

33. A nebulization composition comprising:
(i) tiotropium or a pharmaceutically acceptable salt thereof
(ii) indacaterol or a pharmaceutically acceptable salt thereof,
(iii) sodium chloride,
(iv) tartaric acid and monosodium citrate,
(v) optionally, one or more pharmaceutically acceptable excipients selected from the group consisting of a complexing agent, and a preservative; and
(iv) water for injection.

34. The nebulization composition of claim 1, wherein the composition comprises:
(i) tiotropium or a pharmaceutically acceptable salt thereof
(ii) indacaterol or a pharmaceutically acceptable salt thereof,
(iii) an isotonicity agent,
(iv) a complexing agent, and
(iv) water for injection.

35. A kit for the treatment, prevention or amelioration or one or more symptoms of a disease or disorder associated with bronchoconstriction, comprising
(i) a nebulizer; and
(ii) a nebulization composition according to claim 1.

36. The kit according to claim 35, wherein the nebulizer is selected from a jet nebulizer, ultrasonic nebulizer, vibrating mesh nebulizer and a breath actuated nebulizer.

37. A method of treating relief of bronchospasm in a subject suffering from asthma or chronic obstructive pulmonary disorder comprising administering a nebulizable composition according to claim 1.
